(19) United States
(12) Patent Application Publication
(10) Pub. No.: US 2019/0290475 A1
(43) Pub. Date: Sep. 26, 2019

(54) REVERSIBLE HYSTEROSCOPIC STERILIZATION

(71) Applicants: Albert CHIN, Palo Alto, CA (US); NVISION MEDICAL CORPORATION, San Bruno, CA (US)

(72) Inventor: Albert CHIN, Palo Alto, CA (US)

(21) Appl. No.: 16/302,338

(22) PCT Filed: Apr. 30, 2017
(86) PCT No.: PCT/US2017/030322

§ 371 Date: Nov. 16, 2018

Related U.S. Application Data
(60) Provisional application No. 62/340,636, filed on May 24, 2016.

(51) Int. Cl. A61F 6/22; A61B 17/12

(52) U.S. Cl. CPC A61F 6/225; A61B 1/018; A61B 17/12136; A61B 17/1204

(57) ABSTRACT

An implant device is includes an expandable balloon configured to expand in response to internal pressure applied thereto. A flexible tether is fixed to an end of the expandable balloon, and the flexible tether is buckled and packed into the expandable balloon causing the expandable balloon to expand.

### FIELD OF THE INVENTION

The present invention relates to devices, instruments and methods for surgical procedures. More particularly, the present invention provides devices, instruments and methods for reversible implantation procedures.

### BACKGROUND OF THE INVENTION

Sterilization procedures for women are called tubal sterilization. Tubal sterilization involves closing off the fallopian tubes. Tubal sterilization prevents the egg from moving down the fallopian tube to the uterus and prevents the sperm from reaching the egg.

Hysteroscopic sterilization is a type of tubal sterilization procedure that uses the body's natural openings to place small implants into the fallopian tubes. These implants cause tissue growth that blocks the tubes. No surgical incision is needed. Typically, heretofore, hysteroscopic sterilization involves inserting a tiny device into each fallopian tube with a hysteroscope. The hysteroscope is an instrument that is inserted through the vagina and cervix and then into the uterus. It allows the inside of the uterus and the tubal openings to be seen. Once the devices are in place, scar tissue forms around them. Typically these procedures are irreversible.

Essure®, provided by Bayer, is a commercially available hysteroscopic sterilization device that can be used to perform permanent, irreversible, hysteroscopic sterilization. Another example of an irreversible implant that can be implanted hysteroscopically is provided by Brenzel et al. in US Patent Application Publication No. 20150075536.

US Patent Application Publication No. 20150007827 to Ozdil et al. discloses an occlusion device that includes an openable channel to facilitate reversible female contraception. A tubular device is implanted in the fallopian tube to provide blockage to perform a sterilization function. To reverse, proximal and distal caps of the device are punctured and/or removed to open a channel through the device. However, the device remains implanted in the fallopian tube, even after the reversal procedure is completed.

There is a continuing need for sterilization techniques that are minimally invasive and that are reversible so as to substantially restore the patient to the same condition as before the sterilization was performed.

There is a continuing need for sterilization techniques that do not require a drug regimen or other treatment to affect the biochemistry of the patient and which are reversible.

There is a continuing need for relatively inexpensive techniques for performing reversible sterilization.

### SUMMARY OF THE INVENTION

According to one aspect of the present invention, an implant device is provided that includes: an expandable balloon configured to expand in response to internal pressure applied thereto; a flexible tether fixed to an end of the expandable balloon; and the flexible tether is buckled and packed into the expandable balloon causing the expandable balloon to expand.

In at least one embodiment, the expandable balloon, when expanded by the packing by the flexible tether, expands to a size preventing the expandable balloon from passing through a utero-tuberal junction of a Fallopian tube with a uterus.

In at least one embodiment, a free end of the flexible tether extends proximally of the expandable balloon.

In at least one embodiment, the device further includes a proximal component attached to the expandable balloon, wherein the proximal component is relatively non-expandable.

In at least one embodiment, the proximal component is dimensioned to prevent passage through a proximal os leading to a Fallopian tube from a uterus.

In at least one embodiment, the proximal component comprises a distal portion of a catheter.

In at least one embodiment, the proximal component is tubular.

In at least one embodiment, upon unpacking the tether from the expandable balloon, the expandable balloon resiliently returns to a non-expanded configuration.

In another aspect of the present invention, an apparatus is provided that includes: a catheter having a distal end portion and a proximal end portion; an evertable balloon having a balloon proximal end portion attached to the distal end portion of the catheter and a closed balloon distal end; a flexible tether attached to the balloon distal end; and an inflation port; wherein the apparatus is sealed to allow pressurization thereof sufficient to evert the evertable balloon.

In at least one embodiment, prior to pressurization of the apparatus, the evertable balloon extends into the catheter so that the balloon distal end is proximal of the balloon proximal end portion.

In at least one embodiment, the proximal end portion of the catheter comprises a first cross-sectional dimension and the distal end portion of the catheter comprises a second cross-sectional dimension, wherein the second cross-sectional dimension is smaller than the first cross sectional dimension.

In at least one embodiment, the first cross-sectional dimension is substantially constant over a length of the proximal end portion of the catheter and the second cross-sectional dimension is substantially constant over a length of the distal end portion of the catheter.

In at least one embodiment, the proximal end portion of the catheter comprises a first tubular structure having a first inside diameter and a first outside diameter and the distal end portion of the catheter comprises a second tubular structure having a second inside diameter and a second outside diameter, and wherein the first inside diameter is greater than the second outside diameter.

In at least one embodiment, the distal and proximal end portions of the catheter are separate components.

In at least one embodiment, the apparatus further includes a connecting sleeve attached to and connecting the distal and proximal end portions of the catheter.

In at least one embodiment, the connecting sleeve is severable and is designed to sever upon application of a tensile force greater than a predetermined tensile force.

In at least one embodiment, the apparatus further includes a detachment tube slidably received within the proximal end portion of the catheter, wherein the detachment tube is configured to be advanced relative to the proximal end portion of the catheter to apply a tensile force greater than the predetermined tensile force to the connecting sleeve to sever the connecting sleeve and detach the distal end portion of the catheter from the proximal end portion of the catheter.

In at least one embodiment, the apparatus further includes a seal at an interface between the proximal end portion of the catheter and the detachment tube configured to allow pressurization of the catheter.

In at least one embodiment, the tether extends through the catheter and proximally out of the proximal end portion of the catheter.

In at least one embodiment, the apparatus further includes a support tube extending through at least a portion of the proximal end portion of the catheter and being slidable relative thereto, wherein the tether extends through the support tube and proximally thereof.

In at least one embodiment, the apparatus further includes a support tube seal configured to form a seal between the support tube and the tether to allow pressurization of the catheter.

In at least one embodiment, the support tube seal is adjustable to allow sliding of the tether relative thereto.

In at least one embodiment, the apparatus further includes a support tube extending through at least a portion of the detachment tube and being slidable relative thereto, wherein the tether extends through the support tube and proximally thereof.

In at least one embodiment, the apparatus further includes a detachment tube seal at an interface between the detachment tube and the support tube configured to allow pressurization of the catheter.

In at least one embodiment, the apparatus further includes a sheath surrounding at least a portion of the catheter, the sheath being slidable relative to the catheter to extend distally past a distal end of the catheter to protect the evertable balloon when at least partially everted.

In at least one embodiment, the apparatus is inserted into a working channel of a hysteroscope.

In another aspect of the present invention, a system for removal of an expandable balloon is provided that includes: an expandable balloon configured to expand in response to internal pressure applied thereto; a flexible tether fixed to an end of the expandable balloon; and the flexible tether buckled and packed into the expandable balloon causing the expandable balloon to expand; a free end of the flexible tether extending proximally of the expandable balloon; a first catheter having a proximal end an a distal end; a snare extending through the first catheter and having a working end extending distally of the distal end of the first catheter, configured to capture the flexible tether; and a second catheter in which the first catheter is slidably received; wherein capture of the flexible tether by the snare and iteratively sliding the first catheter relative to the second catheter unpacks the flexible tether from the expandable balloon.

In at least one embodiment, the system further includes a proximal component attached to the expandable balloon, wherein the proximal component is relatively non-expandable, and a distal end of the second catheter is dimensioned to abut the proximal component during the iterative sliding of the first catheter relative to the second catheter.

In at least one embodiment, the tether passes through the proximal portion.

In at least one embodiment, the proximal portion is tubular and the proximal portion and the second catheter have equal or nearly equal cross-sectional dimensions.

In at least one embodiment, the system further includes a hysteroscope having a working channel, the first and second catheters being insertable into the working channel.

In another aspect of the present invention, a method of implanting a device includes: pressurizing an apparatus containing an evertable balloon to at least partially evert the balloon so that at least a portion of the balloon extends distally of a distal end of a catheter of the apparatus; advancing s sheath over the catheter so that the sheath covers the extending balloon; contacting a distal end of the sheath to target tissue surrounding an opening into which the balloon is to be inserted; advancing the balloon through the opening and into a tubular structure; fully everting the evertable balloon when it is not already fully everted; packing the balloon with a flexible tether to expand the balloon; and removing the sheath and catheter, leaving the expanded, packed balloon in place.

In at least one embodiment, the opening is a proximal os and the tubular structure is a Fallopian tube.

In at least one embodiment, the apparatus is inserted through a hysteroscope prior to the contacting.

In at least one embodiment, the advancing comprises sliding the catheter distally relative to the sheath.

In at least one embodiment, the fully everting comprises advancing a support tube inside the catheter relative to the catheter to push the balloon into full eversion.

In at least one embodiment, the method further includes depressurizing the apparatus prior to the packing.

In at least one embodiment, the packing comprises iteratively sliding a support tube through which the tether passes proximally and distally relative to the catheter; during distal sliding, fixing a portion of the tether at a proximal end location of the support tube; and during proximal sliding, releasing fixation of the tether relative to the support tube.

In at least one embodiment, after the balloon has been packed, the method includes advancing a detachment tube within the catheter and contacting the detachment tube against at least one of a distal portion of the catheter and a connecting sleeve joining the distal portion of the catheter with a proximal portion of the catheter with force sufficient to rupture the connection sleeve.

In at least one embodiment, the method further includes cutting the tether at a length leaving a tether tail extending proximally of the distal portion of the catheter.

In at least one embodiment, the removing comprises removing the apparatus and the hysteroscope, while leaving the balloon, tether and distal portion of the catheter in place.

In at least one embodiment, a distal end portion of the catheter remains fixed to the balloon after the removing; wherein the expanded balloon is sufficiently expanded to prevent migration of the balloon through the utero-tubal junction; and wherein a proximal component attached to the balloon has a cross-sectional dimension that prevents migration of the proximal component through the proximal os.

In at least one embodiment, the proximal component is a distal end portion of the catheter that has been detached from a proximal end portion of the catheter.

In another aspect of the present invention, a method of implanting a device includes: pressurizing an apparatus containing an evertable balloon to at least partially evert the balloon so that at least a portion of the balloon extends distally of a distal end of a catheter of the apparatus; advancing the balloon through an opening and into a tubular structure of a body; packing the balloon with a flexible tether to expand the balloon; and removing the catheter, leaving the expanded, packed balloon in place.

In another aspect of the present invention, a method of removing an implant includes: capturing a tether tail extending proximally of an expanded balloon having been expanded by packing the tether therein; drawing the tether tail proximally to unpack the tether from the balloon, thereby allowing the balloon to return to an unexpanded configuration having a smaller dimension than when expanded; and removing the tether and the balloon.

In at least one embodiment, the implant is a sterilization implant and the removing reverses sterilization.

In at least one embodiment, the expanded balloon is implanted in a Fallopian tube and, in the expanded configuration, prevents migration of the expanded balloon through the utero-tubal junction.

In at least one embodiment, the capturing comprises inserting a removal catheter apparatus to align a working end of a snare with the tether tail and so that the tether tail passes the working end; and operating a snare catheter through which the snare passes so that the working end moves proximally relative to the snare catheter to secure the tether tail relative to the snare catheter.

In at least one embodiment, retracting the snare catheter and tether unpacks the expanded balloon.

In at least one embodiment, further retraction of the tether removes the balloon.

In at least one embodiment, a proximal component is attached to the expanded balloon, the method further comprising contacting the proximal component with an outer catheter prior to the drawing the tether tail.

In at least one embodiment, a proximal component is attached to the expanded balloon, the method further comprising contacting the proximal component with an outer catheter through which the snare catheter is slidable.

In at least one embodiment, the method further includes operating the outer catheter and snare catheter through a working channel of a hysteroscope.

In another aspect of the present invention, a method of making a medical apparatus includes: attaching a proximal end portion of a balloon to a distal end portion of a catheter; inverting the balloon into the distal end portion of the catheter; attaching a tether to a distal end portion of the balloon; and releasably connecting the distal end portion of the catheter with a proximal end portion of the catheter.

In at least one embodiment, the releasably connecting comprises attaching a connecting sleeve to a proximal end portion of the distal end portion of the catheter and to a distal end portion of the proximal end portion of the catheter.

In at least one embodiment, the connecting sleeve is configured to rupture upon application of force thereto, to separate the distal end portion of the catheter from the proximal end portion of the catheter.

In at least one embodiment, the method further includes installing a detachment tube in the proximal end portion of the catheter, wherein the detachment tube is slidable in the proximal end portion of the catheter to apply force to sever the distal end portion of the catheter from the proximal end portion of the catheter.

In at least one embodiment, the method further includes installing a support tube slidable within the detachment tube and which slidably receives the tether.

These and other features of the invention will become apparent to those persons skilled in the art upon reading the details of the devices, apparatus and methods as more fully described below.

### BRIEF DESCRIPTION OF THE DRAWINGS

In the course of the detailed description to follow, reference will be made to the attached drawings. These drawings show different aspects of the present invention an, where appropriate, reference numerals illustrating like structures, components, materials and/or elements in different figures are labeled similarly. It is understood that various combinations of the structures, components, materials and/or elements, other than those specifically shown, are contemplated and are within the scope of the present invention.

FIG. 1 is a schematic illustration of the human female reproductive system.

FIG. 2 schematically illustrates a longitudinal sectional view of a system configured to perform hysteroscopic sterilization according to an embodiment of the present invention.

FIG. 3 shows events that may be carried out in a method for implanting a sterilization device according to an embodiment of the present invention.

FIGS. 4A-4H schematically illustrate use of the apparatus of FIG. 2 is performing events useful for a stereoscopic sterilization procedure according to an embodiment of the present invention.

FIG. 5 schematically illustrates an implant device having been implanted according to an embodiment of the present invention.

FIGS. 6A-6F illustrate techniques for making an apparatus according to an embodiment of the present invention.

FIGS. 7A-7D schematically illustrate an apparatus and its use in reversing a sterilization procedure according to an embodiment of the present invention.

FIG. 8 shows events that may be carried out in reversing a sterilization procedure according to an embodiment of the present invention.

### DETAILED DESCRIPTION OF THE INVENTION

Before the present devices, apparatus and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a tube” includes a plurality of such tubes and reference to “the filament” includes reference to one or more filaments and equivalents thereof known to those skilled in the art, and so forth.

The publications discussed herein are provided solely for their disclosure prior to the filing date of the present application. The dates of publication provided may be different from the actual publication dates which may need to be independently confirmed.

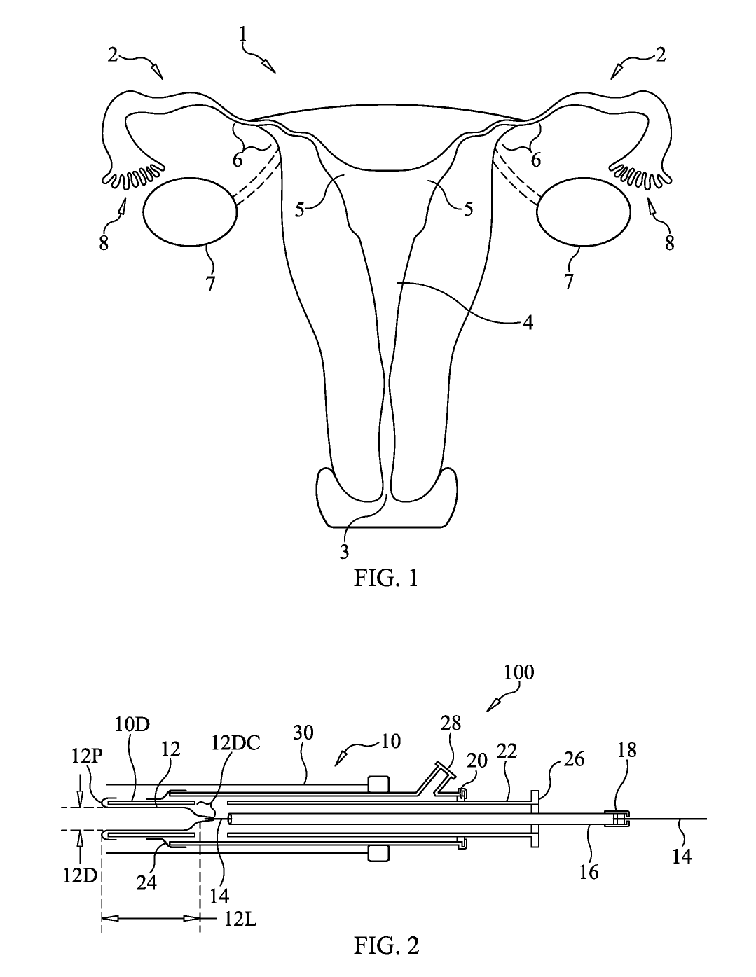

Referring now to FIG. 1, a schematic illustration of the human female reproductive system 1 is shown for reference purposes. Access to the fallopian tubes 2 by the apparatus of the present invention is gained by insertion through the cervix 3 and uterus 4. The proximal os 5 is the opening through the inner wall of the uterus 4 that connects to the Fallopian tube 2 (one each side, respectively), via the utero-tubal junction 6. The ovaries 7 are positioned for release of ova into the fimbria 8 for travel through the fallopian tubes 2 and into the uterus 4.

FIG. 2 schematically illustrates a longitudinal sectional view of an apparatus 100 configured to perform hysteroscopic sterilization according to an embodiment of the present invention. It is noted that although the specific embodiments described herein are directed specifically to reversible sterilization of a human female, that the present invention is not necessarily limited to such application. Other applications include, but are not limited to: reversible hysteroscopic sterilization of the human male (e.g., as the vas deferens); reversible hysteroscopic sterilization of other female mammalian species; reversible hysteroscopic sterilization of other male mammalian species; and reversible blockage of other tubular features in the human or nonhuman mammalian species.

Apparatus 100 is configured to be inserted through a working channel of a hysteroscope for hysteroscopic implantation of a sterilization device in the fallopian tube 2, the utero-tubal junction 6 and ending at the proximal os 5. Apparatus 100 includes an everting balloon catheter 10. Balloon 12 has a length 12L having a value in a range from about 3 to 7 cm, typically about 4 to 6 cm, most typically about 5 cm. The inner diameter 12D of the balloon 12 as shown in FIG. 2 has a value in a range from about 0.7 to 1.2 mm, typically about 0.8 to 1.0 mm, most typically about 0.9 mm. The wall thickness of the balloon 12 typically has a value in the range from about 0.0035 to 0.1000 mm, typically from about 0.0050 to 0.0080 mm and in at least one embodiment, is about 0.0064 mm.

Catheter 10 includes a proximal portion 10P and a distal portion 10D, where the proximal portion 10P has a larger cross-sectional dimension than the distal portion 10D. The proximal portion 10P, in one embodiment has a 4 French (1.33 mm) inside diameter and the distal portion 10D in the same embodiment has a 3 French (1.0 mm) inside diameter. It is noted that the present invention is not limited to these dimensions, as they may vary, as long as the proximal portion 10P has a larger inside diameter than the outside diameter of the distal portion 10D. The length of catheter 10 may be a value in a range from about 30 cm to 100 cm, typically 35 cm to 80 cm. In at least one embodiment, the length of catheter 10 (lengths of 10D and 10P as assembled like shown in FIG. 2) was about 40 cm.

The proximal end 12P of the balloon 12 is attached to the distal end of catheter 10 as shown in FIG. 2, such as by adhesive, thermal welding or equivalent fixation means, and the balloon 12 is inverted into the catheter 10. Specifically, the proximal end 12P is attached to the distal end of distal portion 10D of catheter 10. The distal portion 12DL of the catheter (typically about a 2-3 mm length of the balloon 12) is necked down to an outer diameter of approximately 0.2 mm to 0.5 mm, typically about 0.3 mm A flexible tether 14 is fixed inside the necked down distal end portion 12DL of the balloon 12. Typically the flexible tether is sealably bonded inside the necked down end of the balloon. Typically, the flexible tether 14 is a monofilament polypropylene suture strand, although a monofilament made of one or more other materials may be substituted, or a polyfilament tether may be used. Tether 12 has an outside diameter having a value in a range from about 0.15 to about 0.5 mm, typically about 0.25 mm (0.010″). Tether 14 has a length greater than the remainder of the apparatus, so at to extend through the catheter 10 (and the detachment tube 22 inserted in the catheter 10) with a length greater than the catheter 10 plus detachment tube 22 to extend out of the proximal end of the catheter 10 and detachment tube 22.

Additionally, tether 14 extends through a support tube 16 that lies coaxially outside the tether 14, such that tether 14 extend both proximally and distally of the support tube 16, as shown in FIG. 2. The inner diameter of the support tube 16 is a sliding fit with the tether 14. An adjustable seal 18, such as a Tuohy-Borst fitting or the like, lies on the proximal end of the support tube 16, and it seals against the tether 14 to form a fluid-tight seal between the tether 14 and support tube 16. The proximal end of the catheter 10 includes an adjustable seal 20; such as a Tuohy-Borst fitting or other type of seal that seals against the support tube 16 to maintain fluid pressure in the catheter 10. Following placement of the implant device in the Fallopian tube, the seal 20 may be loosened to allow the detachment tube 22 residing inside the catheter to slide forward and release the implant from the remainder of the catheter 10.

Catheter 10, as noted above, includes a relatively short distal portion 10D and a relatively long proximal portion 10P In at least one embodiment, distal portion 10D had a length of about 3 cm of 3 Fr tubing telescoping by about 1 cm into a distal end of the proximal portion 10P comprising a longer length of 4 Fr tubing. A connecting sleeve 24 overlies and fixes the junction of the distal portion 10D and the proximal portion 10P of the catheter 10. Connecting sleeve may be made of heat shrink tubing for example, or may be elastic or otherwise adhered or fixed to the outer surfaces of the proximal 10P and distal 10D portions as shown in FIG. 2. In at least one embodiment, connecting sleeve comprised a short, approximately 7 mm length of ultrathin polymer heat-shrink tubing. The heat-shrink tubing may be composed of polyethylene terephthalate (PET) or other known heat shrink material, with a wall thickness having a value in the range from about 0.00015″ to about 0.00050″, typically about 0.00025″. The connecting sleeve 24 may be heat, friction and/or adhesive bonded to the proximal 10P and distal 10D portions.

When connecting sleeve 24 is attached to the telescoping catheter portions, 10D and 10P as described, it forms a fluid tight seal over the junction of the portions 10P, 10D, enabling, together with the seals 20 and 18, the attached everting balloon 12 to be pressurized. The connecting sleeve 24 also allows the distal portion 10D to be detached from the proximal portion 10D. The connecting sleeve 24 has a tensile strength predetermined to fail when a force is applied by detachment tube 22 to distal portion 10D, as described in more detail below, so as to place the connecting sleeve 24 under tension. The tensile strength of connecting sleeve has a value in a range from about 0.3 kgf to 0.7 kgf, typically about 0.4 kgf to 06. kgf, more typically about 0.5 kgf.

Detachment tube 22 is slidably inserted within the proximal portion 10P of catheter 10 and is configured with a length and cross-sectional dimension to be slid relative to proximal portion 10P so as to contact and apply force to distal portion 10D. When it is desired to release of the balloon 12 and distal portion 10D from the proximal portion 10P, detachment tube 22 is advanced distally relative to proximal portion 10P to push distal portion 10D forward and apply tension to connecting sleeve 24 in an amount greater than the tensile strength of connecting sleeve 24, causing catastrophic failure and disruption of the connecting sleeve 24, thereby separating distal portion 10D from proximal portion 10P.

The proximal end of the detachment tube 22 contains a sliding seal 26 that allows the suture support tube 16 to slide forward while the catheter 10 and detachment tube 22 are pressurized, during a balloon eversion process described below. The sliding seal 26 may be a Tuohy-Borst fitting with the O-ring seal adjusted to allow the detachment tube 22 to be advanced without loss of fluid pressure, or may be another type of seal that allows the detachment tube 22 to be advance without loss of fluid pressure.

An inflation port 28 is attached to or provided in the body of the proximal portion 10P of catheter 10. An outer sheath 30 covers the catheter 10. Outer sheath 30 has an inside diameter that is larger than the outside diameter or proximal portion 10P. In at least one embodiment, the inside diameter of outer sheath 30 was 5 French (1.67 mm). It is noted that the present invention is not limited to this dimensions, as it may vary, as long as the inside diameter of outer sheath 30 is greater than the outside diameter of proximal portion 10P. Outer sheath 30 may be constructed of nylon, polyvinyl chloride, polyethylene, or similar material. Outer 30 sheath covers and protects the balloon 12 as the catheter 10 is introduced through the seal cap on the working channel of the hysteroscope, and outer sheath 30 is positioned in the uterus 4 outside the proximal os 5 of the Fallopian tube 2 during balloon cannulation and eversion, as described below.

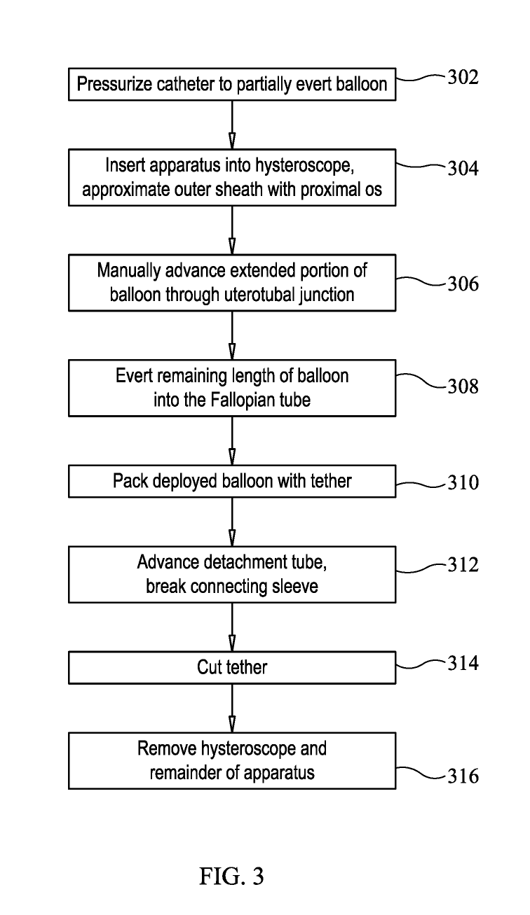

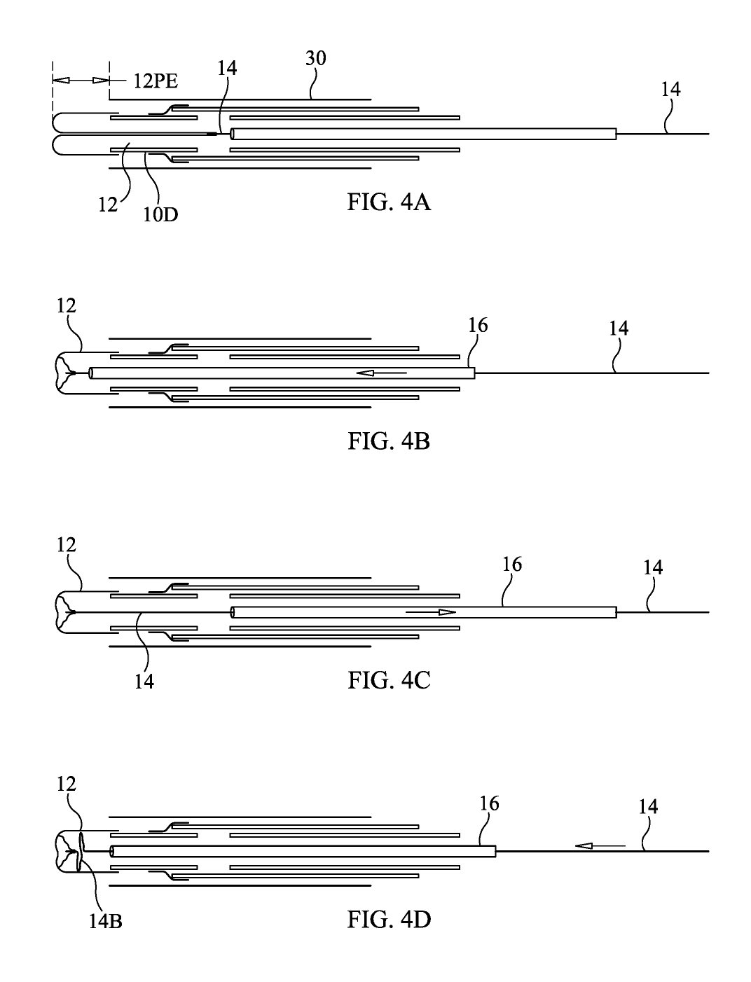

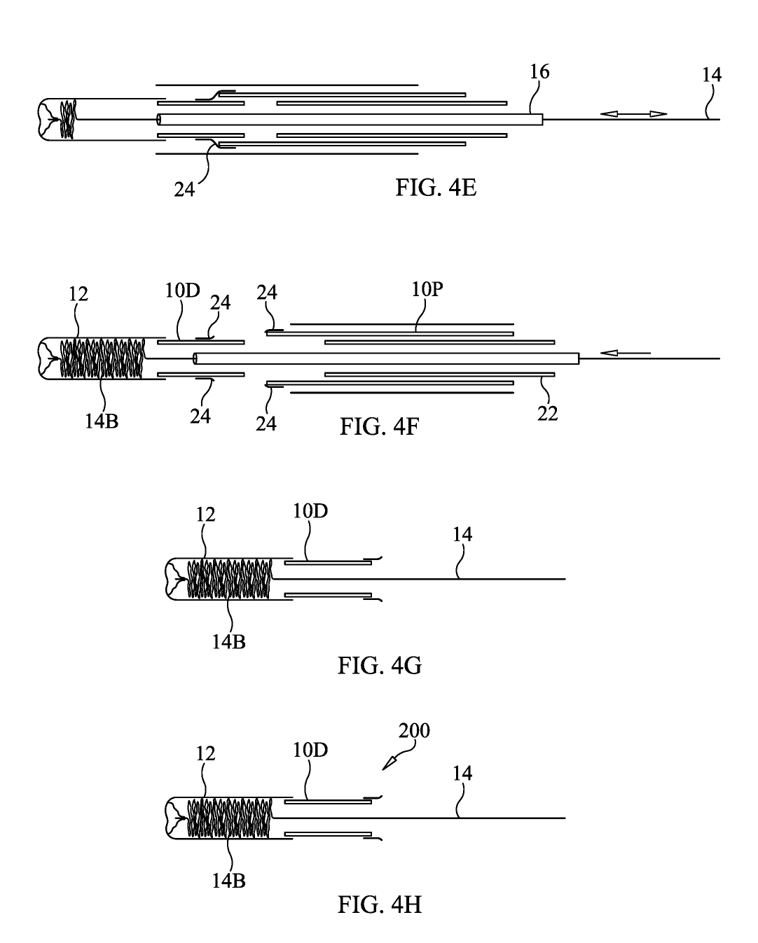

Reference is now made to FIGS. 3 and 4A-4H in describing events that may be carried out in a method for implanting a sterilization device according to an embodiment of the present invention. Apparatus 100 is prepared for insertion into the hysteroscope at event 302. All seals 20, 26 and 18 are sealed and the apparatus 100 is pressurized by applying pressurized fluid through inflation port 28 to partially evert to the balloon 12 by a length 12PE that extends distally of the distal end of catheter portion 10D as shown in FIG. 4A. Length 12PE is typically a value in the range from about 10 mm to about 20 mm, typically about 12 mm to about 18 mm, more typically about 15 mm. The apparatus 100 is pressurized to enable balloon eversion upon advancement of the suture support tube 16. Pressurization is in the range of about 18 atmospheres to about 22 atmospheres, typically about 19 atmospheres to about 21 atmospheres, more typically about 20 atmospheres. The pressurized fluid may be pressurized saline. Alternative pressurized fluids may be substituted or added.

The outer sheath 30 is then advanced forward (distally) to cover the extended portion of the balloon 12. After insertion of a hysteroscope through the cervix 3 and into the uterus 4, and locating the proximal os 5 using the hysteroscope, at event 304, apparatus 100 is advanced through the working channel of the hysteroscope until the distal tip of the outer sheath 30 is placed in gentle contact with the proximal os 5 of the Fallopian tube 2 inside the uterus 4.

While the outer sheath 30 is held stationary, the everted balloon 12 is manually advanced through the utero-tubal junction 6 of the Fallopian tube 2 at event 306. The lumen of the Fallopian tube 2 narrows down to about 0.3-0.5 mm in the utero-tubal junction 6, and this section is extremely difficult to cannulate. Manual advancement of the inflated balloon 12 supported outside the proximal os 5 by the outer sheath 30 has been observed to cross this difficult anatomy in over 90% of cases in a clinical study of 60 patients, using a sheathed everting balloon 12 of the dimensions described above in a Fallopian tube cell collection catheter. The fluid filled balloon inflated to the predetermined pressure as described contains the correct balance of column strength and compliance to cross the utero-tubal junction 6.

Following full manual advancement of the portion 12PE of inflated balloon 12 into the proximal Fallopian tube, the remaining length of the balloon 12 is everted into the Fallopian tube 2 in a controlled fashion at event 308, by advancing the support tube 16 through the sliding seal 26 until it reaches the distal end of the fully everted balloon 12, as schematically illustrated in FIG. 4B.

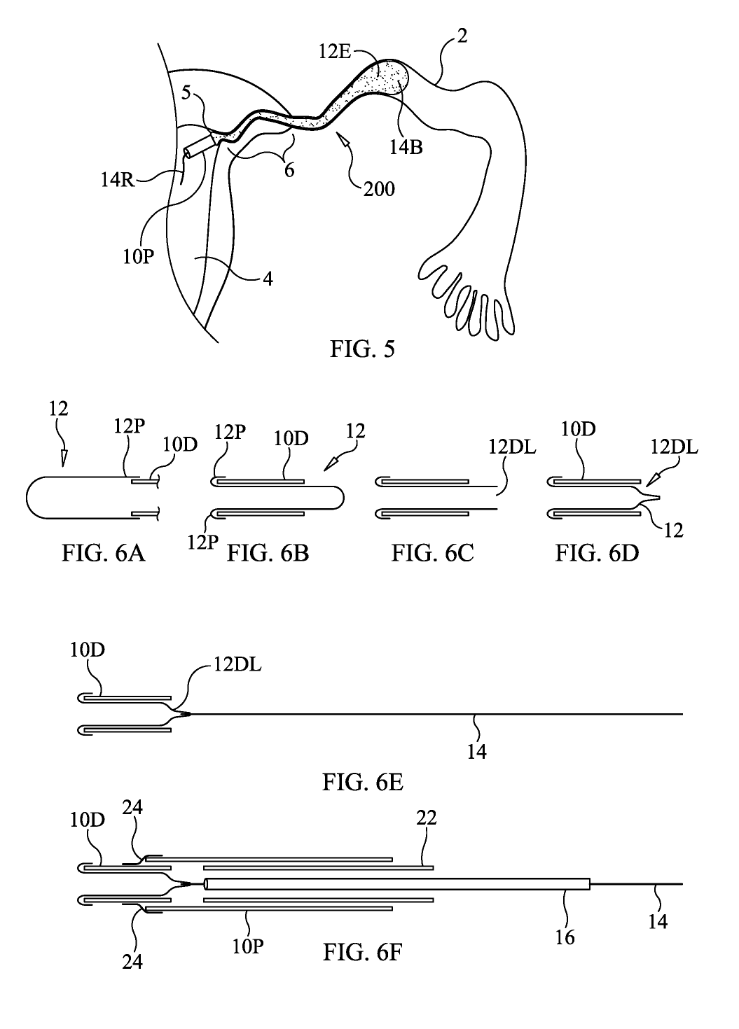

At event 310, the deployed balloon is packed with the tether. The adjustable seal 18 on the proximal end of the support tube 16 is loosened, and the support tube 16 is retracted approximately 2 cm, as illustrated in FIG. 4B. The tether 14 is then fixed relative to the proximal end of the support tube 16, and the support tube 16 is advanced to the end of the balloon 12 as illustrated in FIG. 4D (the support tube 16 is being advanced in FIG. 4D, but has not yet reached the end of the balloon 12), causing the tether 14 to buckle 14B and pack into the distal end of the balloon 12. One non-limiting method of temporarily fixing the tether 14 relative to the support tube 16 is to place a thumb or finger on the tether 14 where it exits the sliding seal 18 on the support tube 16, although other methods can be used, including, but not limited to using a clamp or other temporary fixation means. The tether 14 is released as the support tube 16 is again retracted, and then held fixed as the support tube 16 is re-advanced. The buckled tether 14 is wedged in the distal end of the balloon 12 during packing, and it remains packed as the support tube 16 is retracted. Repetitive suture packing as illustrated in FIG. 4E distends the balloon 12 distal to the utero-tubal junction 6, forming an expanded balloon 12E (see FIG. 5) that cannot contract and migrate into the uterus 4 upon fluid deflation of the balloon 12.

When the balloon 12 is fully tether packed, the adjustable seal 20 on the proximal end of the catheter is loosened, and the detachment tube 22 is pushed distally relative to proximal portion 10P to press against the distal portion 10D with sufficient force to break the connecting sleeve 24 at event 312 and as schematically illustrated in FIG. 4F. By breaking/severing the connecting sleeve as described, the balloon 12 and the distal portion 10D of catheter 10 are separated from the proximal portion 10P and the remainder of the apparatus 100, except for the tether 14, as illustrated in FIG. 4G. The detached implant 200, comprising the balloon 12, the distal portion 10D of the catheter 10 and the packed tether 14B, as illustrated in FIG. 4H, is lodged on both sides of the utero-tubal junction 6, as illustrated in FIG. 5. As a result, the implant device 200 will not dislodge or migrate in either direction. The tether-packed balloon 12 distends the Fallopian tube 2 distal to the utero-tubal junction 6, preventing the implant 200 from migration towards the uterus 4, as the Fallopian tube 2 contains cilia that move in the direction of the uterus 4. The proximal portion 10P in the uterus 4 has a diameter larger than the inside diameter of the proximal os 5, preventing its migration into the Fallopian tube 2. Upon detachment of the implant device 200 from the remainder of the apparatus 100, the tether 14 extending proximally out of the distal portion 10D3 Fr catheter segment is cut using hysteroscopic shears at event 314, so that a residual tether tail 14R remains in the uterus 4, as illustrated in FIG. 5. The residual tail can be used to perform a process of sterilization reversal, as described below. At event 316 the hysteroscope and the remainder of the apparatus 100 contained by the hysteroscope are removed from the uterus and cervix to complete the procedure. The balloon 12 remains implanted inside the Fallopian tube 2, and the distal portion 10D of catheter 10 resides in the uterine cavity outside the proximal os 5 of the Fallopian tube 2.

FIGS. 6A-6E illustrate techniques for making an apparatus 100 according to an embodiment of the present invention. FIG. 6A illustrates the proximal end 12P of the balloon 12 being attached to the distal end portion 10D of catheter 10. As noted above, this attachment may be carried our using adhesive, thermal welding or equivalent fixation means. FIG. 6B shows balloon 12 having been inverted into the distal catheter portion 10D. At FIG. 6C the balloon tip is cut off, leaving an open distal end portion 12DL. The distal end portion 12DL is necked down, such as by shrinking as shown in FIG. 6D. Next the tether 14 is sealed to (preferably within) the distal end portion 12DL which also recloses the distal end of the balloon 12 so that it can be pressurized. Typically the flexible tether 14 is sealably bonded inside the necked down end of the balloon 12. At FIG. 6F the connecting sleeve 24 is connected to catheter portions 10P and 10D to seal the juncture thereof, allowing the catheter to be pressurized. The 22 detachment tube is inserted into the catheter 10 and the support tube 16 is slid over the tether 14 and inserted into the detachment tube 22 and catheter 10.

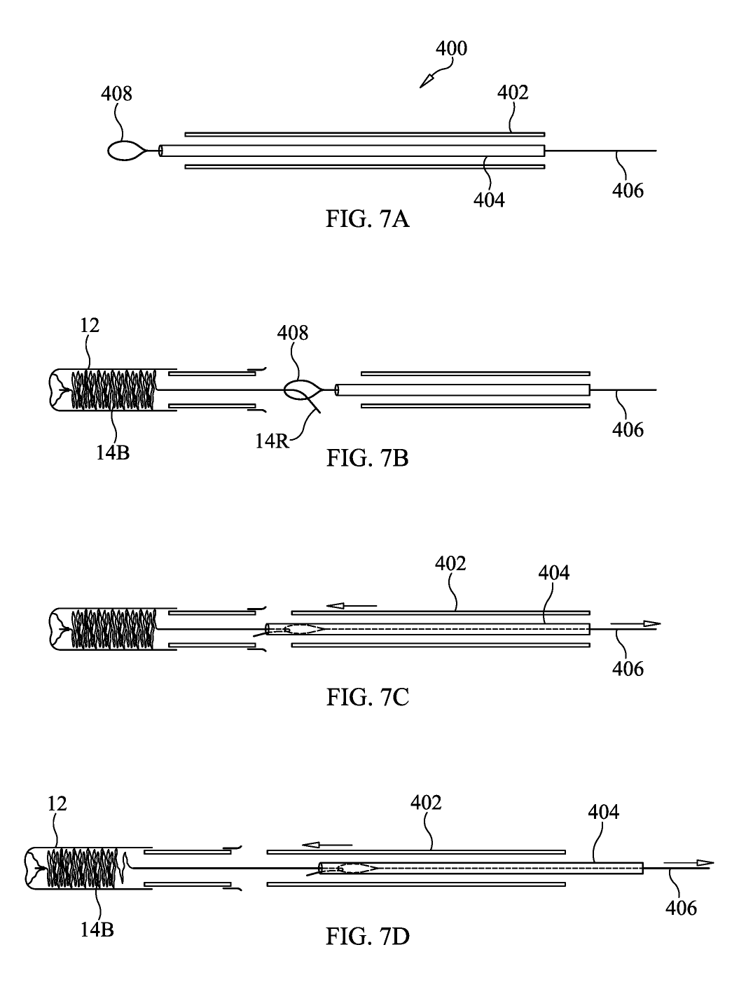

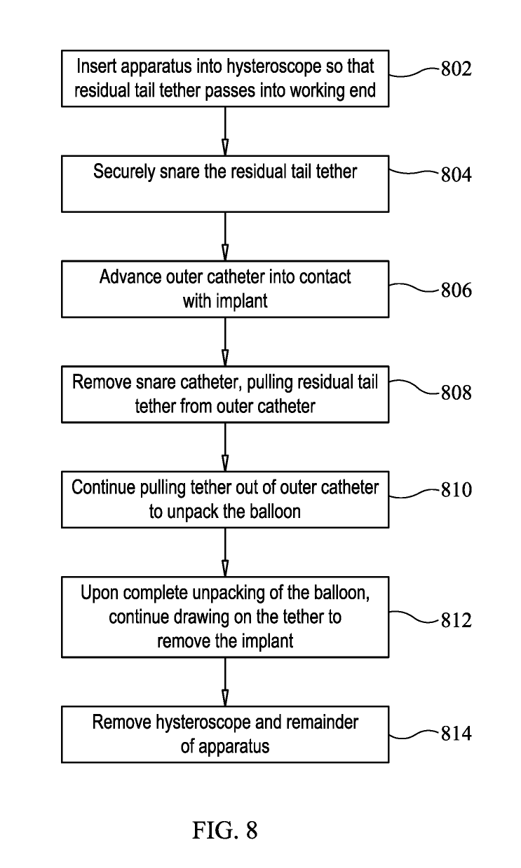

FIGS. 7A-7D schematically illustrate an apparatus 400 and its use in reversing a sterilization procedure according to an embodiment of the present invention and FIG. 8 shows events that may be carried out in reversing a sterilization procedure according to an embodiment of the present invention. An outer catheter 402 that is preferably the same cross-sectional dimensions as that of the distal portion 10D or slightly larger or smaller but still able to contact distal portion 10D and align lumens therewith is provided over a snare catheter 404. In at least one embodiment, outer catheter 402 is approximately 3 Fr in size, but could be slightly larger or smaller. Snare catheter 404 includes a snare 406 slidably inserted therethrough that includes a working end 408 at the distal end thereof.

After insertion of a hysteroscope through the cervix 3 and into the uterus 4, and locating the residual tail tether 14R and distal catheter portion 10D abutting the proximal os 5 using the hysteroscope, at event 802, removal catheter apparatus 400 is advanced through a working channel of the hysteroscope to align the working end 408 of the snare 406 with the residual tail tether 14R, and is further advanced so that the residual tail tether 14R passes through the working end 408 as schematically illustrated in FIG. 7B.

The proximal end portion of the snare 406 is drawn proximally while holding the snare catheter 404 stationary, or the proximal end portion 406 is held stationary while the snare catheter 404 is advanced distally, or the snare catheter 404 is advanced distally while simultaneously drawing the snare proximally to draw the residual tail tether 14R into the snare catheter 404 at event 804 and as schematically illustrate in FIG. 7C, thereby securing the snare of the residual tail tether 14R.

The outer catheter 402 is advanced into contact with the distal catheter portion 10D of the implant device at event 806 and also schematically illustrated in FIG. 7C. While holding the outer catheter 402 in contact with the distal portion 10D, the snare catheter 404 is removed from the outer catheter 402 at event 806 and as schematically illustrated in FIG. 7D, drawing the residual tether 14R with it. The removal of the snare catheter can be performed by pulling only on the snare 406, or by pulling on both the snare 406 and the snare catheter 404, being careful that the snare 406 does not slide distally relative to the snare catheter 404 during the removal process.

At event 810, removal of tether 14 continues by repeatedly drawing the tether 14 out to the catheter 402 to unpack all of the tether out of the balloon 12. Once the balloon 12 has been entirely unpacked, the deflated balloon is then removed from the Fallopian tube 2 by continuing to draw on the tether 14. As the tether is sealed in the distal end of the balloon 12, the deflated balloon 12 everts as it is drawn out of the Fallopian tube 2, and, together with the distal portion 10D, through the uterus 4 and out of the cervix 3 in event 812.

At event 814 the hysteroscope and the remainder of the apparatus 400 contained by the hysteroscope are removed from the uterus 4 and cervix 3 to complete the procedure.

While the present invention has been described with reference to the specific embodiments thereof, it should be understood by those skilled in the art that various changes may be made and equivalents may be substituted without departing from the true scope of the invention as defined by the claims below. In addition, many modifications may be made to adapt a particular situation, material, composition of matter, process, process step or steps, to the objective and scope of the present invention. All such modifications are intended to be within the scope of the present invention.

1-8. (canceled)

9. A device for delivering an implant to a Fallopian tube, comprising:
a catheter having a distal end portion and a proximal end portion;
a balloon having a proximal end portion attached to said distal end portion of said catheter and the balloon having a closed distal end, the balloon being movable between a first, inverted position and a second, everted position; and
a flexible tether attached to said closed distal end of the balloon and extendable through the catheter proximally of the proximal end portion of the catheter;
wherein said flexible tether is packable into the closed distal end of the balloon in the second, everted position, to form the implant, the implant being positionable from a proximal os across a utero-tubal junction into the Fallopian tube.

10-13. (canceled)

14. The device of claim 9, wherein said distal and proximal end portions of said catheter are separable.

15. The device of claim 14, further comprising a connecting sleeve separably connecting said distal and proximal end portions of said catheter.

16. The device of claim 15, wherein said connecting sleeve is severable upon application of a tensile force greater than a predetermined tensile force, such that the distal and proximal end portions of the catheter are separated.

17. The device of claim 16, further comprising a detachment tube slidably received within said proximal end portion of said catheter, wherein said detachment tube is advanceable relative to said proximal end portion of said catheter to apply the tensile force greater than said predetermined tensile force to said connecting sleeve.

18-19. (canceled)

20. The device of claim 9, further comprising a support tube extendable through at least a portion of said proximal end portion of said catheter, and slidable relative thereto, the tether extending through said support tube and proximally thereof.

21. (canceled)

22. The device of claim 20, wherein the flexible tether is selectively slidable relative to the support tube, such that the tether is packable into the distal end of the balloon via buckling.

23-24. (canceled)

25. The device of claim 9, further comprising a sheath surrounding at least a portion of the proximal end of said catheter, said sheath being slidable relative to said catheter to extend distally past the distal end portion of said catheter to protect said balloon when at least partially everted.

26-31. (canceled)

32. A method of delivering an implant from a catheter to a Fallopian tube, said method comprising:
fully everting a balloon into the Fallopian tube, the balloon being movable between a first, inverted position, to a second, everted position;
packing the balloon with a flexible tether; and
removing the sheath and catheter, leaving the packed balloon as the implant positioned across the utero-tubal junction into the Fallopian tube.

33-37. (canceled)

38. The method of claim 32, wherein said packing comprises iteratively sliding a support tube through which the flexible tether passes proximally and distally relative to the catheter;
during distal sliding, fixing a portion of the flexible tether at a proximal end location of the support tube such that the tether is packed into the distal end of the balloon via buckling; and
during proximal sliding, releasing fixation of the flexible tether relative to the support tube such that the packed tether remains in the distal end of the balloon.

39. The method of claim 32, further comprising, subsequent to packing the balloon, separating a distal portion of the catheter from a proximal portion of the catheter, the distal portion of the catheter connected to the packed balloon.

40. The method of claim 39, further comprising cutting the tether at a length leaving a tether tail extending proximally of the distal portion of the catheter.

41-42. (canceled)

43. The method of claim 39, wherein the distal end portion of the catheter is attached to the balloon to form a proximal component of the implant, the packing of the balloon preventing migration of the proximal component through the proximal os from the Fallopian tube.

44. (canceled)

45. A method of removing an implant, said method comprising:
capturing a tail of a tether extending proximally of an everted balloon packed with a buckled portion of the tether from which the tail extends;
drawing the tether tail proximally to unpack the tether and reduce a profile of the balloon, thereby allowing migration of the implant; and
removing the implant.

46. The method of claim 45, wherein the implant is a sterilization implant and said removing reverses sterilization.

47. The method of claim 45, wherein the implant is positioned in a Fallopian tube to prevent migration of the implant through the utero-tubal junction.

48. The method of claim 45, wherein said capturing comprises:
inserting a removal catheter to align a working end of a snare with the tether tail;
manipulating the working end of the snare to secure the tether tail; and
retracting the working end of the snare in a proximal direction to unpack the tether from the balloon.

49. (canceled)

50. The method of claim 48, wherein further retraction of the tether removes the balloon.

51-58. (canceled)

59. The method of claim 32, further comprising:
pressurizing the balloon to at least partially evert the balloon such that at least a portion of the balloon extends distally of a distal end of the catheter;
advancing a sheath over said catheter so that the sheath covers the distally extended balloon portion; and
contacting a distal end of the sheath to a proximal os to advance the balloon across the utero-tubal junction and into the Fallopian tube.

60. The device of claim 20, wherein a support tube seal is configured to form a seal between the support tube and the tether, the support tube seal being selectively adjustable to pack the tether into the closed distal end of the balloon.
